(19) United States
(12) Patent Application Publication
(10) Pub. No.: US 2024/0148552 A1
(43) Pub. Date: May 9, 2024

(54) HIGH FREQUENCY VITRECTOR

(71) Applicant: Alcon Inc., Fribourg (CH)

(72) Inventor: Nathaniel Reyes, Santa Ana, CA (US)

(21) Appl. No.: 18/483,713

(22) Filed: Oct. 10, 2023

Related U.S. Application Data
(60) Provisional application No. 63/382,754, filed on Nov. 8, 2022.

(51) Int. Cl. A61F 9/007; A61B 17/00

(52) U.S. Cl. CPC A61F 9/00763; A61B 17/00; A61B 2017/00544

(57) ABSTRACT

Certain embodiments provide a high-frequency vitrector including a handpiece having an outer tube mounted thereto with an oscillating inner tube within the outer tube and coupled to a drive diaphragm. Extension and retraction pneumatically controlled valves receive pressure from extension and retraction channels and control flow of gas from a pressurized gas source to the extension and retraction sides, respectively, of a chamber containing the drive diaphragm. The handpiece may include an input diaphragm coupled to a valve stem to which the extension and retraction valves are connected. One side of a chamber containing the input diaphragm is coupled to the extension channel and the other to the retraction channel. An aspiration line passes through the input diaphragm and valve stem and has an end in fluid communication with the inner tube.

### TECHNICAL FIELD

The present disclosure relates generally to surgical instruments for performing vitrectomy.

### BACKGROUND

Vitreo-retinal procedures may include a variety of surgical procedures performed to restore, preserve, and enhance vision. Vitreo-retinal procedures may be appropriate to treat many serious conditions of the back of the eye. Vitreo-retinal procedures may treat conditions such as age-related macular degeneration (AMD), diabetic retinopathy and diabetic vitreous hemorrhage, macular hole, retinal detachment, epiretinal membrane, CMV retinitis (Cytomegalovirus retinitis), and many other ophthalmic conditions.

The vitreous is a normally clear, gel-like substance that fills the center of the eye. It may make up approximately two-thirds of the eye's volume, giving it form and shape before birth. Certain problems affecting the back of the eye may require a vitrectomy, or surgical removal of the vitreous. Removal of vitreous can involve a vitrector that works like a tiny guillotine, with an oscillating microscopic cutter to remove the vitreous gel in a controlled fashion. The vitrector is powered by a pneumatic vitrectomy machine, incorporated into a surgical console, including one or more pneumatic valves (also referred to as drive valves).

The vitrector is coupled to the surgical console by first and second channels that may be implemented as separate tubes or different sections of a single tube. The surgical console alternatively couples the first and second channels to a high-pressure source in order to cause the cutter of the vitrector to oscillate. The length of the first and second channels is typically over a meter long and may be up to two meters. As a result, the pressure at the vitrector is significantly attenuated, which increases the time required to build up enough pressure to cause the cutter to change direction.

### BRIEF SUMMARY

The present disclosure relates generally to a vitrector with an integrated valve and direct connection to a high-pressure source in order to achieve higher cutting frequency.

Certain embodiments provide a system for performing vitrectomy including a source of pressurized gas and a pneumatic control system having an extension port and a retraction port. The pneumatic control system is configured to alternately supply gas from the source of pressurized gas to the extension port and the retraction port. The system further includes a vitrector including a handpiece defining an extension channel coupled to the extension port, a retraction channel coupled to the retraction port, and a supply channel coupled to the source of pressurized gas. An outer tube is mounted to the handpiece and defines a side opening. An inner tube is slidably positioned within the outer tube. A drive diaphragm within the handpiece is configured to oscillate the inner tube. An extension pneumatically controlled valve is mounted within the handpiece and is in fluid communication with the supply channel. The extension pneumatically controlled valve is configured to couple the supply channel to an extension side of a drive diaphragm chamber containing the drive diaphragm responsive to pressure applied to the extension channel. A retraction pneumatically controlled valve is mounted within the handpiece and is in fluid communication with the supply channel. The retraction pneumatically controlled valve is configured to couple the supply channel to a retraction side of the drive diaphragm chamber responsive to pressure applied to the retraction channel.

The following description and the related drawings set forth in detail certain illustrative features of one or more embodiments.

### BRIEF DESCRIPTION OF THE DRAWINGS

The appended drawings depict only examples of certain embodiments of the present disclosure and are therefore not to be considered as limiting the scope of this disclosure.

FIG. 1 illustrates a cutter suitable for use with a high-frequency vitrector, in accordance with certain embodiments.

FIG. 2 illustrates a schematic of a pneumatic system for controlling a high-frequency vitrector, in accordance with certain embodiments.

FIG. 3 illustrates a schematic of a high-frequency vitrector, in accordance with certain embodiments.

FIGS. 4A and 4B illustrate a high-frequency vitrector at a distal end and a proximal end of an oscillation cycle, respectively, in accordance with certain embodiments.

FIG. 5 is a diagram illustrating an example handpiece incorporating valves for implementing a high-frequency vitrector, in accordance with certain embodiments

To facilitate understanding, identical reference numerals have been used, where possible, to designate identical elements that are common to the drawings. It is contemplated that elements and features of one embodiment may be beneficially incorporated in other embodiments without further recitation.

### DETAILED DESCRIPTION

While features provided herein may be discussed relative to certain embodiments and figures below, all embodiments discussed herein can include one or more of the advantageous features discussed herein. In other words, while one or more embodiments may be discussed as having certain advantageous features, one or more of such features may also be used in accordance with various other embodiments discussed herein. In similar fashion, while exemplary embodiments may be discussed below as device, instrument, or method embodiments it should be understood that such exemplary embodiments can be implemented in various devices, instruments, and methods.

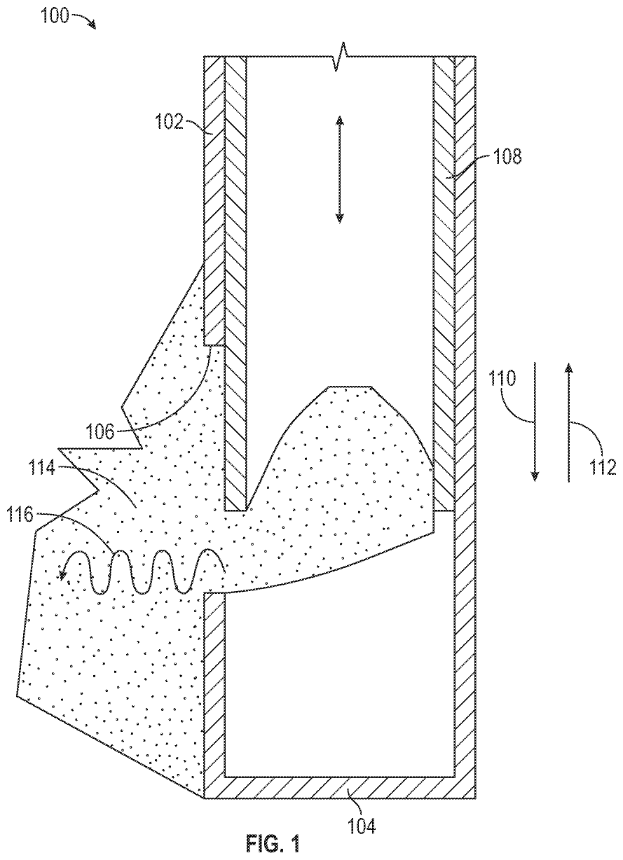

FIG. 1 illustrates a cutter 100 that may be used with a high-frequency vitrector, according to the embodiments disclosed herein. The cutter 100 is exemplary only and any cutter suitable for performing vitrectomy may be used according to the embodiments disclosed herein. The cutter 100 includes an outer tube 102 having a covered distal end 104 and a side opening 106. The side opening 106 extends through a cylindrical wall of the outer tube 102. An inner tube 108 oscillates within the outer tube 102 with a distal direction 110 being defined as movement toward the distal end 104 and a proximal direction 112 being defined as movement away from the distal end 104. The inner tube 108 is coupled to an aspirator, i.e., vacuum pressure. The vacuum draws vitreous 114 into the outer tube 102 through the side opening 106. As the inner tube 108 traverses the side opening 106, it severs a piece of the vitreous, which is then drawn out of the inner tube 108 by the vacuum pressure. The range of motion of the inner tube 108 may be from an extended position in which the inner tube 108 extends completely across the side opening 106 and a retracted position in which the inner tube 108 does not extend over the side opening 106 at all or only partially extends across the side opening 106.

At least three aspects of the operation of the cutter 100 are improved using the vitrector disclosed herein. First, as the oscillation frequency of the inner tube 108 increases, the sizes of the chunks of the vitreous 114 cut by the inner tube 108 become smaller and more frequent, resulting in less force exerted on the surrounding vitreous. In other words, the increased oscillation frequency results in reduced traction of the surrounded vitreous. Second, in certain existing cutters, such as cutter 100, when the inner tube 108 oscillates, some vibration 116 is transmitted to the vitreous, which may cause harm to sensitive tissue, such as the retina. By increasing the frequency of oscillation of the inner tube 108, using the embodiments disclosed herein, the frequency of the vibration 116 is increased, resulting in more rapid attenuation. Third, as friction between the inner tube 108 and the outer tube 102 may be unavoidable, the embodiments disclosed herein allow for applying more force to the inner tube 108 thereby enabling higher frequency oscillation.

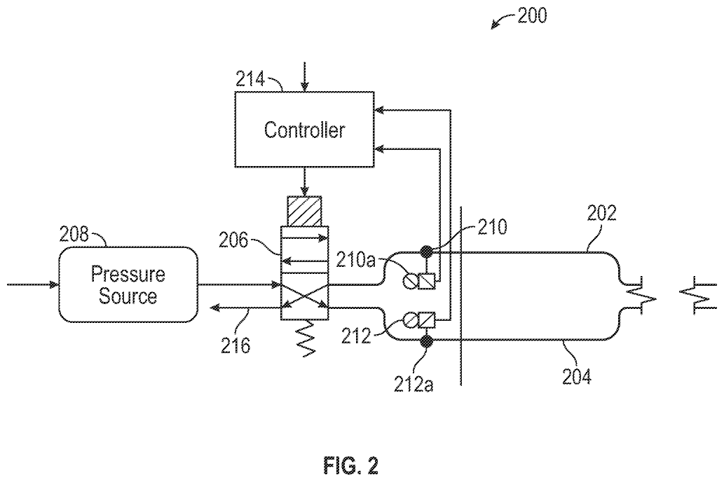

FIG. 2 illustrates a schematic of a pneumatic system 200 for controlling a high-frequency vitrector, according to the embodiments disclosed herein. The components of the pneumatic system 200 may be incorporated inside a surgical console. The pneumatic system 200 controls pressure of high-pressure gas supplied to an extension channel 202 and a retraction channel 204 at a user-specified, default, or automatically selected pressure. The pneumatic system 200 alternates between supplying high-pressure gas to the extension channel 202 and a retraction channel 204 at a user-specified, default, or automatically selected frequency. For example, the high-pressure gas may be received from a wall outlet of a typical medical facility, which can have a pressure of between 58 and 120 psi (pounds per square inch) (4 to 8.3 bar). The pneumatic system 200 is exemplary only. The high-frequency vitrector disclosed herein has the advantage of being useable with a pneumatic system according to any approach known in the art without requiring modification thereof.

The example pneumatic system 200 includes a pneumatic valve 206 coupling a pressure source 208 (e.g., a regulated pressure source such as a gas cylinder or a wall outlet gas supply) to output port 210 and output port 212. The output port 210 is coupled to the extension channel 202 and the output port 212 is coupled to the retraction channel 204. Pressure sensors 210a, 212a may sense the pressure of gas supplied to the output ports 210, 212, respectively and provide pressure measurements to the controller 214 to enable the controller 214 to regulate pressure supplied to the output ports 210, 212.

The extension channel 202 and retraction channel 204 may be embodied as multi-channel tube or separate tubes. In some embodiments, the pneumatic valve 206 may be controlled by controller 214. The controller 214 itself may control the pneumatic valve 206 according to control parameters such as desired cut rate and/or duty cycle. In some embodiments, the pressure of the pressure source 208 may also be regulated by controller 214 or a separate controller (e.g., internal to a surgical console). The controller 214 may regulate pressure (e.g., to balance between lower pressures for reducing gas consumption and higher pressures for faster cut rates and/or to increase a dynamic range of available cut rates).

The pneumatic valve 206 may include a solenoid that operates to move the pneumatic valve 206 to one of the two positions as directed by control signals from controller 214. In an extended position, pneumatic valve 206 may allow pressurized gas to pass through pneumatic valve 206 to output port 210 to provide high-pressure gas to the extension channel 202 while venting pressurized gas from output port 212 through an exhaust port 216. In a retracted position, the pneumatic valve 206 may provide pressurized gas to output port 212 and vent pressurized gas from the output port 210 through the exhaust port 216.

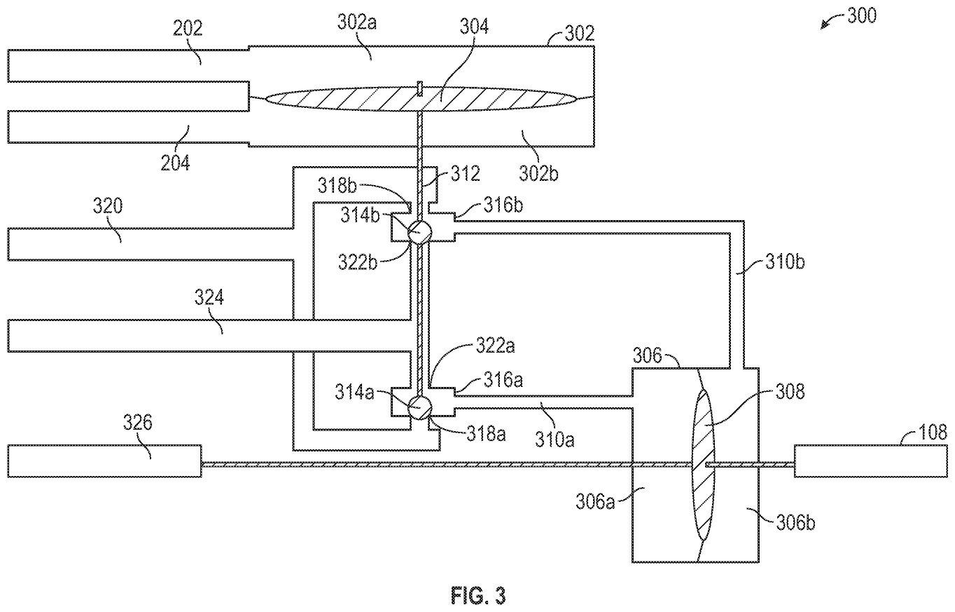

FIG. 3 illustrates an example embodiment of a high-frequency vitrector 300. The high-frequency vitrector 300 includes a plurality of pneumatic components, e.g., valves and diaphragms, that may be mounted within a handpiece and used to amplify alternating pressure received from the extension channel 202 and the retraction channel 204 using gas received from the pressure source 208 in order to drive oscillation of the inner tube 108 within the outer tube 102, the outer tube 102 being mounted to the handpiece.

The high-frequency vitrector 300 includes an input diaphragm chamber 302 divided into two sides by an input diaphragm 304. An extension side 302a of the input diaphragm chamber 302 is connected to the extension channel 202 and a retraction side 302b of the diaphragm chamber is connected to the retraction channel 204. The high-frequency vitrector 300 further includes a drive diaphragm chamber 306 divided into two sides by a drive diaphragm 308. An extension side 306a of the drive diaphragm chamber 306 is connected to an extension drive channel 310a and a retraction side 306b of the drive diaphragm chamber 306 is connected to a retraction drive channel 310b. An aspiration line 326 coupled to a source of vacuum pressure may pass through the drive diaphragm 308 and connect to the inner tube 108.

The input diaphragm 304 is connected by a coupler 312 to an extension valve 314a and a retraction valve 314b. The coupler 312 is a mechanical coupling that causes the extension valve 314a and retraction valve 314b to change position responsive to movement of the input diaphragm 304. As such, the coupler 312 may be a rigid body to which the valves 314a, 314b are secured, a push-pull cable or cable system, or any mechanical coupling known in the art.

The extension valve 314a is positioned within an extension valve chamber 316a having a vent valve surface 318a connected to a vent line 320. The extension valve chamber 316a further has a supply valve surface 322a connected to a supply line 324. The extension valve chamber 316a is further coupled to the extension drive channel 310a. The supply line 324 may be connected to the pressure source 208 or a different pressure source. The gas supplied to the supply line 314 may be regulated to the same pressure or a different pressure from the pressure supplied to the output ports 210, 212.

The retraction valve 314b is positioned within a retraction valve chamber 316b having a vent valve surface 318b connected to the vent line 320. The retraction valve chamber 316b further has a supply valve surface 322b connected to the supply line 324. The retraction valve chamber 316b is further coupled to the retraction drive channel 310b.

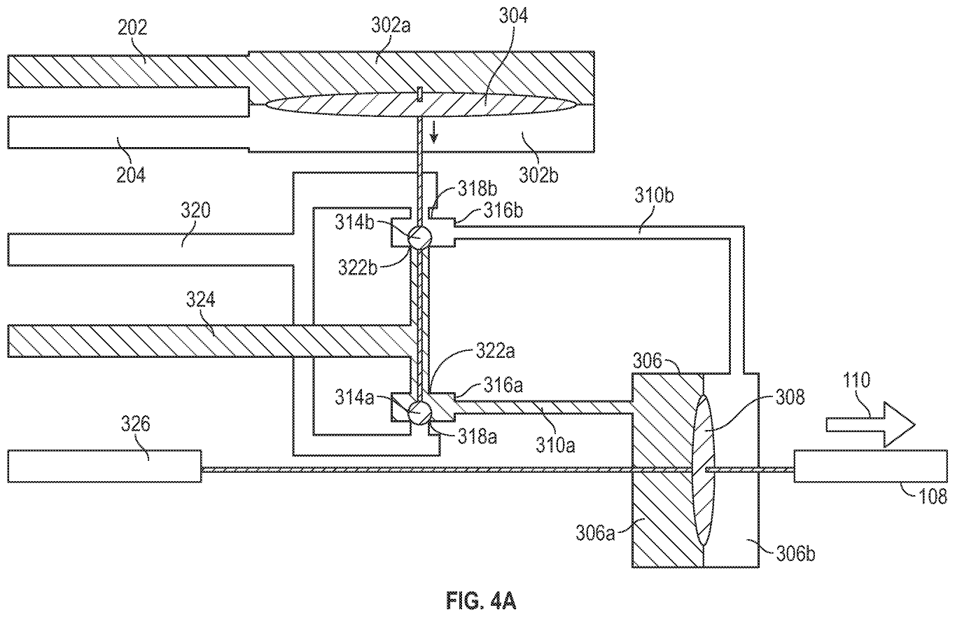

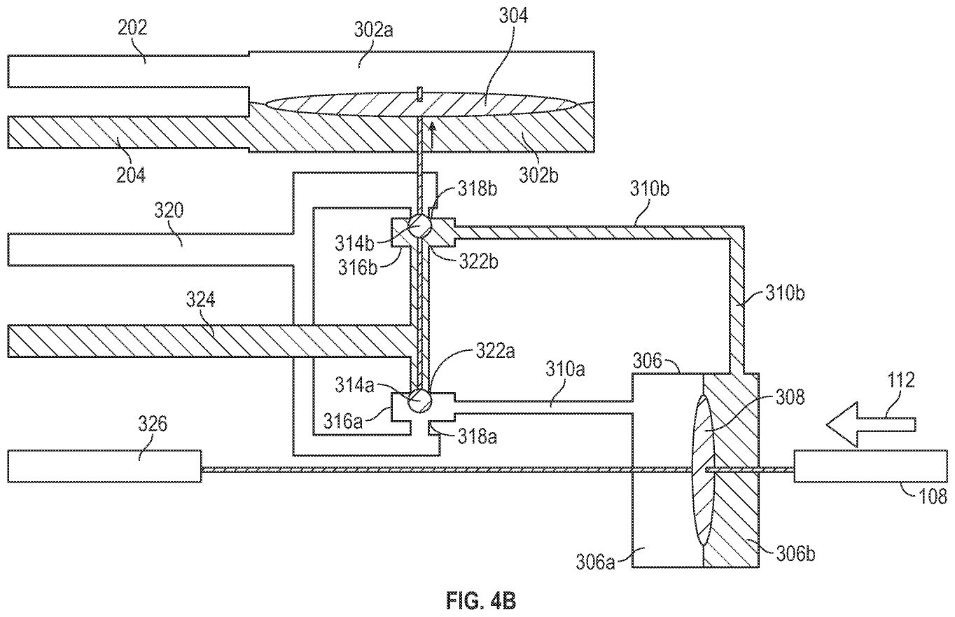

FIGS. 4A and 4B illustrate the different states of operation of the high-frequency vitrector 300. As used in the following description closing a valve surface 318a, 318b, 322a, 322b may be understood as substantially closing a valve surface such that gas flow through the valve surface is substantially reduced, e.g., by at least 80 percent, at least 90 percent, or at least 99 percent of gas flow when the valve surface 318a, 318b, 322a, 322b is open.

Referring specifically to FIG. 4A, in an extended state (i.e., the inner tube 108 being driven to or being in the extended position), the extension channel 202 is connected to the pressure source 208, thereby pressurizing the extension side 302a of the input diaphragm chamber 302. In this state, the input diaphragm 304 is displaced such that the coupler 312 drives the extension valve 314a toward the vent valve surface 318a and drives the retraction valve 314b toward the supply valve surface 322b. The vent valve surface 318b and the supply valve surface 322a are therefore open. In the extended state, with the supply valve surface 322a open, the supply line 324 is connected to the extension side 306a of the drive diaphragm chamber 306 by way of the valve chamber 316a and the extension drive channel 310a. The drive diaphragm 308 is therefore displaced in the distal direction 110 and drives the inner tube 108 in the distal direction 110.

In the extended state, the retraction side 306b of the drive diaphragm chamber 306 is connected to the vent line 320 by way of the valve chamber 316b due to opening of the vent valve surface 318b thereby allowing gas to vent from the retraction side 306b into the vent line 320.

Referring specifically to FIG. 4B, in a retracted state (i.e., the inner tube being driven to or being in the retracted position), the retraction channel 204 is connected to the pressure source 208, thereby pressurizing the retraction side 302b of the input diaphragm chamber 302. In this state, the input diaphragm 304 is displaced such that the coupler 312 drives the retraction valve 314b toward the vent valve surface 318b and drives the extension valve 314a toward the supply valve surface 322a thereby closing the vent valve surface 318b and the supply valve surface 322a. The vent valve surface 318a and the supply valve surface 322b are therefore open. In the retracted state, with the supply valve surface 322b open, the supply line 324 is connected to the retraction side 306b of the drive diaphragm chamber 306 by way of the valve chamber 316b and the retraction drive channel 310b. The drive diaphragm 308 is therefore displaced in the proximal direction 112 and drives the inner tube 108 in the proximal direction 112.

In the retracted state, the extension side 306a of the drive diaphragm chamber 306 is connected to the vent line 320 by way of the valve chamber 316a due to opening of the vent valve surface 318a thereby allowing gas to vent from the extension side 306a into the vent line 320.

The vitrector 300 oscillates the valves 314a, 314b and diaphragms 304, 308 between the extended state and the retracted state. The gas flow through the extension channel 202 and the retraction channel 204 need only pressurize the sides 302a, 302b of the input diaphragm chamber 302 sufficient to change the positions of the valves 314a, 314b, which is much less than the force required to overcome friction and inertia of the inner tube 108. The supply line 324 may be maintained at a constant pressure and gas from the supply line 324 need only traverse a relatively short path through one of the valve chambers 316a, 316b and either the extension drive channel 310a or the retraction drive channel 310b. The force applied to the inner tube 108 may therefore be much greater and switched at a much higher speed.

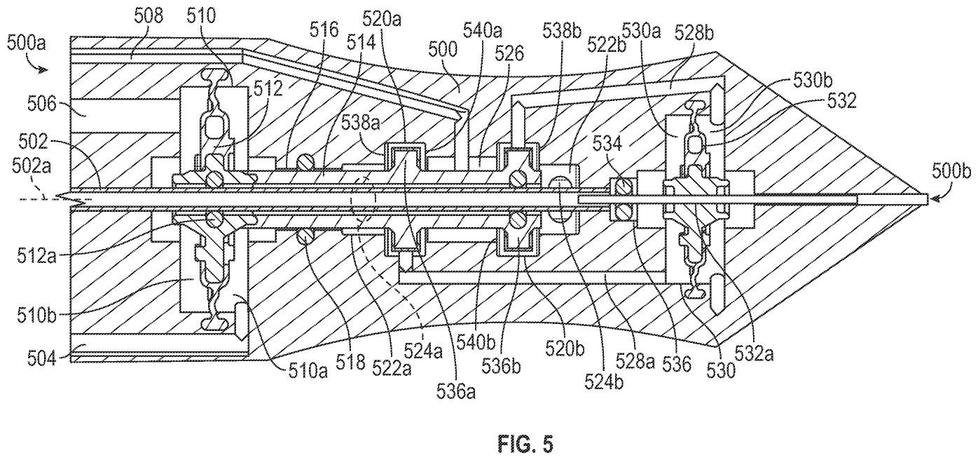

FIG. 5 is a diagram illustrating an example handpiece 500 incorporating components for implementing a high-frequency vitrector, in accordance with certain embodiments. The handpiece 500 is described below starting from the proximal end 500a of the illustrated portion of the handpiece 500 to the distal end 500b.

The handpiece 500 includes an aspiration tube 502. The aspiration tube 502 may be cylindrical in shape, e.g. a cylindrical tube, and various other components of the handpiece 500 may be substantially (e.g., within 1 to 3 mm (millimeters)) centered on a central axis 502a of the aspiration tube 502. In use, the aspiration tube 502 is connected to a source of vacuum pressure. The handpiece 500 further defines an extension channel 504 that is coupled to the extension channel 202 during use and defines a retraction channel 506 that is connected to the retraction channel 204 during use. The handpiece 500 defines a supply channel 508 that is connected to the pressure source 208 or other source of pressurized gas during use. In the illustrated embodiment, the aspiration tube 502, extension channel 504, retraction channel 506, and supply channel 508 extend toward the proximal end 500a of the handpiece 500 parallel to one another, though other arrangements are possible.

The handpiece 500 defines an input diaphragm chamber 510 having an input diaphragm 512 positioned therein and dividing the input diaphragm chamber 510 into an extension side 510a and a retraction side 510b. The input diaphragm 512 may be substantially centered on the central axis 502a and be generally symmetrical about the central axis 502a. The aspiration tube 502 passes through the input diaphragm 512 and a seal 512a incorporated into the input diaphragm 512 may permit sliding of the input diaphragm 512 relative to the aspiration tube 502 while hindering gas flow through the interface between the input diaphragm 512 and the aspiration tube 502. Oscillation of the diaphragm may be substantially (e.g., within 2 degrees of) parallel to the central axis 502a. The extension side 510a of the diaphragm chamber is connected to the extension channel 504 and the retraction side 510b is connected to the retraction channel 506.

The input diaphragm 512 is connected to a valve stem 514 such that the valve stem 514 oscillates in response to oscillation of the input diaphragm 512. The valve stem 514 may be substantially centered on the central axis 502a and be symmetrical about the central axis 502a. The valve stem 514 extends from the input diaphragm 512 through a valve stem channel 516. A seal 518 may be positioned within the valve stem channel 516 and hinder gas flow through the valve stem channel 516.

The valve stem 514 passes through an extension valve chamber 520a and a retraction valve chamber 520b. The valve stem 514 likewise passes through an extension vent chamber 522a, a retraction vent chamber 522b, and a supply chamber 526. Each vent chamber 522a, 522b includes a vent opening 524a, 524b that vents gas out of the handpiece 500 either directly to the atmosphere or through an exhaust tube. The supply chamber 526 is in fluid communication with the supply channel 508 and positioned between the valve chambers 520a, 520b. The extension vent chamber 522a is in fluid communication with the valve chamber 520a and is positioned between the valve chamber 520a and the valve stem channel 516. The retraction vent chamber 522b is in fluid communication with the valve chamber 520b with the valve chamber 520b between the retraction vent chamber 522b and the supply chamber 526.

This arrangement is exemplary only. For example, supposing the supply chamber 526 was connected to a vent opening whereas the vent chambers 522a, 522b were connected to the supply channel 508 and lacked the vents 524a, 524b. In this case, oscillation would still be possible but in the opposite direction (pressurizing the extension channel 504 would cause movement of the inner tube 108 in the proximal direction 112 and pressurizing the retraction channel 506 would cause movement in the distal direction 110).

The extension valve chamber 520a is connected by extension drive channel 528a to an extension side 530a of a drive diaphragm chamber 530 having a drive diaphragm 532 positioned therein. The retraction valve chamber 520b is connected by retraction drive channel 528b to a retraction side 530b of the drive diaphragm chamber 530.

The inner tube 108 passes through the drive diaphragm 532 and a seal 532a incorporated into the drive diaphragm 532 permits sliding of the inner tube 108 relative to the drive diaphragm while hindering gas flow between the extension side 530a and the retraction side 530b through the drive diaphragm 532. The inner tube 108 may pass through another seal 534 positioned within a cavity 536 in the extension side 530a and into the aspiration tube 502 while remaining slidable within the aspiration tube 502.

An extension valve 536a is positioned in the extension valve chamber 520a and a retraction valve 536b is positioned in the retraction valve chamber 520b. Each valve chamber 520a, 520b has a vent side 538a, 538b and a supply side 540a, 540b, respectively. Each valve chamber 520a, 520b is connected to the corresponding vent chamber 522a, 522b through an opening in the vent side 538a, 538b, respectively. Each valve chamber 520a, 520b is connected to the supply chamber 526 through an opening in the supply side 540a, 540b, respectively. When pressed against the supply side 540a, 540b, each valve 536a, 536b substantially seals the valve chamber 520a, 520b from the supply chamber 526. When pressed against the vent side 538a, 538b, each valve 536a, 536b can substantially seal the valve chamber 520a, 520b from the high the corresponding vent chamber 522a, 522b. Portions of the valves 536a, 536b in contact with the sides 538a, 538b, 540a, 540b may be covered with a material to enhance sealing, such as an elastomeric material.

In the illustrated implementation, the supply sides 540a, 540b face inwardly toward one another and the supply chamber 526. The vent sides 538a, 538b face outwardly from one another and toward the vent chambers 522a, 522b. Surfaces of the valves 536a, 536b that contact the vent sides 538a, 538b and the supply sides 540a, 540b may be covered with a sealing material, such as an elastomer in order to promote sealing.

In the extended state, pressurized gas is supplied to the extension channel 504, driving the input diaphragm toward the distal end 500b (the distal direction 110). This pushes the valve stem 514 and valves 536a, 536b such that the extension valve 536a is pushed against the vent side 538a of the extension valve chamber 520a and away from the supply side 540a. In this state, the extension valve chamber 520a is in fluid communication with the supply chamber 526 and substantially sealed from the extension vent chamber 522a, allowing gas from the pressure source 208 or other source of pressurized gas to flow through the extension drive channel 528a and into the extension side 530a. This urges the drive diaphragm 532 and inner tube 108 in the distal direction 110.

In the extended state, the retraction valve 536b is pushed against the supply side 540b and away from the vent side 538b. In this state, the retraction valve chamber 520b is in fluid communication with the retraction vent chamber 522b and substantially sealed from the supply chamber 526, placing the retraction side 530b in fluid communication with the vent 524b and allowing pressurized gas to exit the retraction side 530b.

In the retracted state, pressurized gas is applied to the retraction channel 506 driving the input diaphragm toward the proximal end 500a (the proximal direction 112). This pushes the valve stem 514 and valves 536a, 536b such that the retraction valve 536b is pushed against the vent side 538b of the retraction valve chamber 520b and away from the supply side 540b. In this state, the retraction valve chamber 520b is in fluid communication with the supply chamber 526 and substantially sealed from the retraction vent chamber 522b, allowing pressurized gas from the pressure source 208 or other source of pressurized gas to flow through the retraction drive channel 528b and into the retraction side 530b. This urges the drive diaphragm 532 and inner tube 108 in the proximal direction 112.

In the retracted state, the extension valve 536a is pushed against the supply side 540a and away from the vent side 538a. In this state, the extension valve chamber 520a is in fluid communication with the extension vent chamber 522a and substantially sealed from the supply chamber 526, placing the extension side 530a in fluid communication with the vent 524a and allowing pressurized gas to exit the retraction side 530b.

As is apparent in FIG. 5 and from the foregoing description, the extension valve chamber 520a and retraction valve chamber 520b remain in fluid communication with the extension drive channel 528a and retraction drive channel 528b regardless of the state of the valves 536a, 536b, respectively.

The foregoing description is provided to enable any person skilled in the art to practice the various embodiments described herein. Various modifications to these embodiments will be readily apparent to those skilled in the art, and the generic principles defined herein may be applied to other embodiments. Thus, the claims are not intended to be limited to the embodiments shown herein, but are to be accorded the full scope consistent with the language of the claims.

What is claimed is:

1. A vitrector comprising:
an outer tube defining a side opening;
an inner tube positioned within the outer tube;
a handpiece defining an extension channel, a retraction channel, and a supply channel, the outer tube being mounted to the handpiece; and
a plurality of pneumatic components within the handpiece and configured to amplify alternating pressure supplied to the extension and the retraction channel using gas supplied through the supply channel to drive oscillation of the inner tube within the outer tube.

2. The vitrector of claim 1, wherein the plurality of pneumatic components within the handpiece comprise:
a drive diaphragm chamber;
a drive diaphragm within the drive diaphragm chamber and configured to oscillate an inner tube of a cutter;
an extension pneumatically controlled valve mounted within the handpiece and in fluid communication with a supply channel, the extension pneumatically controlled valve configured to couple the supply channel to an extension side of a drive diaphragm chamber containing the drive diaphragm responsive to pressure applied to the extension channel; and
a retraction pneumatically controlled valve mounted within the handpiece and in fluid communication with the supply channel, the retraction pneumatically controlled valve configured to couple the supply channel to a retraction side of the drive diaphragm chamber responsive to pressure applied to the retraction channel.

3. The vitrector of claim 2, wherein:
the handpiece defines at least one vent opening;
the extension pneumatically controlled valve is further configured to couple the at least one vent opening to the extension side of the drive diaphragm chamber responsive to pressure applied to the retraction channel; and
the retraction pneumatically controlled valve is further configured to couple the at least one vent opening to the extension side of the drive diaphragm chamber responsive to pressure applied to the extension channel.

4. The vitrector of claim 3, further comprising an input diaphragm within an input diaphragm chamber defined by the handpiece, the extension channel in fluid communication with an extension side of the input diaphragm chamber and the retraction channel being in fluid communication with a retraction side of the input diaphragm chamber, the input diaphragm being connected to the extension pneumatically controlled valve and the retraction pneumatically controlled valve by a coupler.

5. The vitrector of claim 4, wherein the coupler is a valve stem, the extension pneumatically controlled valve and the retraction pneumatically controlled valve being secured to the valve stem.

6. The vitrector of claim 7, further comprising an aspiration tube, the aspiration tube passing through the input diaphragm, valve stem, and drive diaphragm, a distal end of the aspiration tube being in fluid communication with the inner tube

7. The vitrector of claim 5, wherein the handpiece defines an extension valve chamber and a retraction valve chamber, the extension pneumatically controlled valve being positioned within the extension valve chamber and the retraction pneumatically controlled valve being positioned within the retraction valve chamber, the extension valve chamber being in fluid communication with the extension side of the drive diaphragm chamber and the retraction valve chamber being in fluid communication with the retraction side of the drive diaphragm chamber.

8. The vitrector of claim 7, further comprising a supply chamber positioned between the extension valve chamber and the retraction valve chamber and in fluid communication with the extension valve chamber and the retraction valve chamber, the valve stem passing through the supply chamber, the supply chamber in fluid communication with the supply channel.

9. The vitrector of claim 8, wherein:
the at least one vent opening is an extension vent opening and a retraction vent opening;
the handpiece defines an extension vent chamber in fluid communication with the extension vent opening and a retraction vent chamber in fluid communication with the retraction vent opening, the extension valve chamber positioned between the extension vent chamber and the supply chamber and the retraction valve chamber positioned between the retraction vent chamber and the supply chamber.

10. The vitrector of claim 9, further comprising a first seal extending around the valve stem and positioned between the input diaphragm and the extension vent chamber.

11. The vitrector of claim 10, further comprising a second seal extending around the valve stem and positioned between the retraction vent chamber and the drive diaphragm.

12. The vitrector of claim 2, wherein the inner tube is slidably positioned within the outer tube, and wherein the inner tube is connected to the drive diaphragm such that the inner tube oscillates responsive to oscillation of the drive diaphragm.

13. A system for performing vitrectomy comprising:
a source of pressurized gas;
a pneumatic control system having an extension port and a retraction port, the pneumatic control system configured to alternately supply gas from the source of pressurized gas to the extension port and the retraction port; and
a vitrector including:
a handpiece defining an extension channel coupled to the extension port, a retraction channel coupled to the retraction port, a supply channel coupled to the source of pressurized gas, and a drive diaphragm chamber;
an outer tube mounted to the handpiece and defining a side opening;
an inner tube slidably positioned within the outer tube
a drive diaphragm positioned within the drive diaphragm chamber and configured to oscillate the inner tube;
an extension pneumatically controlled valve mounted within the handpiece and in fluid communication with the supply channel, the extension pneumatically controlled valve configured to couple the supply channel to an extension side of a drive diaphragm chamber containing the drive diaphragm responsive to pressure applied to the extension channel; and
a retraction pneumatically controlled valve mounted within the handpiece and in fluid communication with the supply channel, the retraction pneumatically controlled valve configured to couple the supply channel to a retraction side of the drive diaphragm chamber responsive to pressure applied to the retraction channel.

14. The system of claim 13, wherein:
the handpiece defines at least one vent opening;
the extension pneumatically controlled valve is further configured to couple the at least one vent opening to the extension side of the drive diaphragm chamber responsive to pressure applied to the retraction channel; and
the retraction pneumatically controlled valve is further configured to couple the at least one vent opening to the extension side of the drive diaphragm chamber responsive to pressure applied to the extension channel.

15. The system of claim 14, further comprising an input diaphragm within an input diaphragm chamber defined by the handpiece, the extension channel in fluid communication with an extension side of the input diaphragm chamber and the retraction channel being in fluid communication with a retraction side of the input diaphragm chamber, the input diaphragm being connected to the extension pneumatically controlled valve and the retraction pneumatically controlled valve by a valve stem.
